(19) United States
(12) Patent Application Publication
(10) Pub. No.: US 2011/0263011 A1
(43) Pub. Date: Oct. 27, 2011

(54) ANTIFOULING COATING COMPOSITION COMPRISING FUNCTIONALIZED NANOPARTICLES

(76) Inventors: Jun Qiu, Weert (NL); Ronnie B.M. de Rijk, Nuth (NL); Jens Christoph Thies, Eijsden (NL)

(21) Appl. No.: 13/127,104

(22) PCT Filed: Oct. 30, 2009
(86) PCT No.: PCT/EP09/64407

§ 371 Date: Jul. 18, 2011

(51) Int. Cl. C12M 1/40; C12M 3/00; B01L 3/00; B05D 3/02; B05D 3/06; B32B 15/04; B32B 27/36; B32B 27/30; B32B 27/06; B32B 27/34; B32B 25/20; B32B 17/06; C09D 5/16; B32B 27/32

(52) U.S. Cl. 435/304.1; 523/122; 522/148; 435/305.1; 422/551; 422/502; 422/549; 422/501; 422/527; 427/385.5; 427/508; 428/523; 428/480; 428/412; 428/522; 428/500; 428/473.5; 428/422; 428/475.5; 428/447; 428/426; 428/457

(57) ABSTRACT

Method for providing a substrate with an anti-biofouling coating the method comprising: a. obtaining a coating composition comprising nanoparticles being grafted with reactive groups and hydrophilic polymer chains and a solvent; b. applying the coating composition to the substrate; and c. optionally curing the coating composition herein the surface tension of the coating composition at 25° C. is below 40 mN/m.

The invention relates to a method for providing a substrate with an anti-biofouling coating, a substrate containing the anti-biofouling coating and a molded article containing the anti-biofouling coating.

Coating compositions for the suppression or prevention of biofouling are well known. Objects in contact with water, especially those made of synthetic materials, are generally prone to suffering from an undesirable accumulation of biologically derived organic species, be it from protein adsorption, bacterial adsorption and subsequent spreading, or thrombosis. This is commonly termed ‘biofouling’. Biofouling can have serious consequences. For example, in the medical area bacterial infections via catheters may be caused by biofouling and in industry the clogging of filters, accumulation of organic material on surfaces etc also causes problems.

The use of disposable products in the life sciences and medical fields (e.g. blood collection tubes, microtitre plates, microfluid devices, biosensors, cell culture flasks and dishes, microtubes, PCR tubes, separation filters, pipette tips etc.) has grown tremendously in the past decades. These disposables are made from a variety of materials such a polypropylene, polyethylene terephthalate, polystyrene etc. and offer many advantages. For example they are stable, easily sterilized, and versatile. In addition, the can be easily processed, have good gas barrier properties, high impact resistance, good optical transparency and are can be mass produced relatively cheaply. However, these disposables can also suffer problems with biofouling.

There have been solutions proposed for the problem of biofouling but these solutions can have unexpected effects on the sensitive assays and tests that the samples are subjected to. For example, Vacutainer® SST™ blood collection tubes are coated with a surfactant, Silwet™ L-720 an organosilane surfactant which reduces the biofouling. However, this surfactant has been shown to cause interference with certain assays, for example, by desorbing capture antibodies from the solid phase used in the Immulite total triiodothyronine immunoassay. A fuller discussion of the numerous assays affected can be found in Becton-Dickenson Technical Bulletin VS7313 and Clin Chim Acta 2007; 378 (1-2): 181-193. A further example of a non-biofouling product is the Corning NBS Microplate (Catalog #3676). This product also uses a surfactant to provide non-biofouling properties and this surfactant can also affect the results of subsequent assays. It is hypothesized that the non-biofouling surfactants are leaching out of the coatings and in to the samples where they affect the results of subsequent tests.

Therefore, there exists a need for an anti-biofouling solution that avoids significant interference with samples and testing. The coatings described in WO2006/016800 seem to provide such a solution. These coatings have good anti-biofouling performance but do not contain surfactants that leach into samples and affect assays. However, such coatings were difficult to apply successfully to different substrates and, in particular, to the different geometries. For example, when the coatings of '800 where applied to blood collection tubes the coatings were cracked, incomplete, prone to peeling, and did not provide acceptable performance.

It would be advantageous to provide a coating method that gave good coverage of the substrate for these biological disposables. Given their relatively small size and difficult geometries this is not straightforward. Additionally it would be desirable that the coating method provide consistent, quality coatings that adhere to the substrate.

Surprisingly it has been found that good, consistent, anti-biofouling coatings can be manufactured if the surface tension of the coating composition is below a certain level. While not wishing to be bound by theory it is believed that when the surface tension of the coating composition is low the problems associated with the geometry and size of the disposables are reduced or eliminated.

A further advantage is that the resulting coating can show good mechanical properties, like hardness and scratch resistance.

Yet another advantage is that the coating can show good anti-fogging properties.

Yet a further advantage is that the coating can show a good adhesion to substrates.

Yet a further advantage is that the coating can show have good lubricious properties.

Yet a further advantage is that the coating can be designed to be bioreactive, by grafting specific groups to the surface of the particles, or incorporating them in the network formed by said reactive particles.

Yet another advantage of the coating is it can have optical clarity, especially in the dry state.

The present invention relates to a method for providing a substrate with an anti-biofouling coating the method comprising:
a. obtaining a coating composition comprising nanoparticles being grafted with reactive groups and hydrophilic polymer chains and a solvent;
b. applying the composition to the substrate; and
c. optionally curing the coating
wherein the surface tension of the coating composition is at 25° C. is below 40 mN/m.

The coating composition has a surface tension at 25° C. lower than 40 mN/m, more preferably lower than 30 mN/m. The surface tension of the coating composition preferably is higher than 10 mN/m. Surface tensions of materials are known from literature or can be measured by, for example, ASTM D 1331-89 (2001).

Preferably the coating compositions herein have a weight percentage of solids in the composition of from 2 wt % to 7 wt %.

Particles

The coating composition may comprise all kind of particles, as long as the particles are grafted with the reactive groups and the hydrophilic polymer chains. It is possible that the coating composition comprises organic and/or inorganic particles. Examples of organic particles are carbon nano tubes or carbon nano spheres. Preferably the coating composition comprises inorganic particles, because in this way a very strong coating is obtained. The average largest diameter of the particles is preferably less than 100 nm, still more preferably less than 50 nm. Thus, the coating composition contains nanoparticles. This is because this provides a very strong coating, having a smooth surface. It is also possible with particles of these very small diameters to provide a transparent coating.

In the case of spherical particles there is only one diameter to consider, so that the diameter is equal to the smallest diameter. For non-spherical particles (for instance but not limited to rods and platelets) the largest diameter is measured as the largest straight line drawn across the particle. Methods for determining the particle dimensions include optical microscopy, scanning microscopy and atomic force microscopy (AFM). If a microscopical method is used the dimensions of 100 randomly chosen particles are measured and the average is calculated. Examples of suitable inorganic particles are particles that comprise SiO₂, TiO₂, ZnO₂, TiO₂, SnO₂, Am—SnO₂, ZrO₂, Sb—SnO₂, Al₂O₃., Au or Ag. Preferably, the particles are nanoparticles and the nanoparticles comprise SiO₂.

Hydrophilic Polymers

It is possible that the particles are grafted with all kind of hydrophilic polymer chains. A hydrophilic polymer chain is a polymer chain that dissolves in water at least one temperature between 0 and 100° C. Preferably a polymer is used that dissolves in water in a temperature range between 20 and 40° C. Preferably the hydrophilic polymer dissolves for at least 0.1 gram per litre of water, more preferably for at least 0.5 grams per litre, most preferably for at least 1.0 gram per litre. For determining the solubility in water the polymer chains are taken not comprising the groups for grafting the polymer chains or any other group that is attached to the polymer after the polymerisation, for example an ionic group. Preferably the solubility is determined in water having a pH of between 3 and 10, more preferably in between 5.5 and 9, most preferably having a pH of 7.

The polymer chain may comprise one monomer species (homopolymer), or more species (copolymer) arranged in a random manner or in ordered blocks.

Preferably the hydrophilic polymer chains comprise monomer units of ethylenoxide, (meth)acrylic acid, (meth)acrylamide, vinylpyrrolidone, 2-hydroxyethyl(meth)acrylate, phosphorylcholine, glycidyl(meth)acrylate or saccharides.

One of the typical advantages that the coating imparts to the coated object are very good anti-biofouling properties of the coating, resulting from the hydrophilicity of the polymer chain. These properties increase with increasing concentration and length of hydrophilic polymer chain at the surface of the coating.

Preferably the chains of the hydrophilic polymer comprise at least an average of 5 monomeric units, more preferably the polymer comprises at least an average of 7 monomeric units, still more preferably the polymer comprises at least an average of 10 monomeric units, most preferably the polymer comprises at least an average of 15 monomeric units.

The concentration may for example be increased by increasing the density of grafted polymers to the particles, increasing the length, or by increasing the weight ratio of the particles in the coating composition.

For obtaining good anti-fogging properties polymer chains having a relatively short length are preferred.

Another advantage of the coating composition is a low static water contact angle. Preferably the static water contact angle is below 50°, more preferably below 40°, still more preferably below 30°.

Groups Used for Grafting

Groups for grafting the hydrophilic polymer chains and compounds comprising the reactive groups to the particles may comprise all groups known in the art for grafting, for instance but not limited to (trialkoxy)silanes, thiols, amines, silane hydrides. Due to the grafting reaction the hydrophilic polymer chains and the compounds comprising the reactive groups are chemically bounded to the surface of the particles. It is possible that the hydrophilic polymers and the compounds comprising the reactive group comprise more than one group for grafting per molecule. In a more preferred embodiment the hydrophilic polymers and the compounds reactive groups have on average one group for grafting per molecule. In case of the hydrophilic polymer the group for grafting preferably is an endgroup attached to the chain of the hydrophilic polymer.

Reactive Groups.

As reactive groups, groups are used that may react with the substrate and/or react to form a cross-linked phase so to form a coating comprising the particles. It is possible that a single species of reactive groups is used, able to mutually react, for example in a homo polymerisation reaction. Examples of such reactive groups include acrylate and methacrylate groups. Another possibility is that a mixture of groups is used, for example groups that are able to react in a copolymerisation reaction. Examples of such groups include carboxylic acids and/or carboxylic anhydrides combined with epoxies, acids combined with hydroxy compounds, especially 2-hydroxyalkylamides, amines combined with isocyanates, for example blocked isocyanate, uretdion or carbodiimide, epoxies combined with amines or with dicyandiamides, hydrazinamides combined with isocyanates, hydroxy compounds combined with isocyanates, for example blocked isocyanate, uretdion or carbodiimide, hydroxy compounds combined with anhydrides, hydroxy compounds combined with (etherified) methylolamide (“amino-resins”), thiols combined with isocyanates, thiols combined with acrylates or other vinylic species (optionally radical initiated), acetoacetate combined with acrylates, and when cationic crosslinking is used epoxy compounds with epoxy or hydroxy compounds. Addition reactions such as 2+2 photo cyclo addition and 4+2 thermal additions are also possible.

Preferably the reactive groups are selected from acrylates, methacrylates, epoxy, vinyl ethers, allyl ethers, styrenics, or combinations thereof.

It is also possible that reactive groups are attached to the hydrophilic polymer chains, however preferably at least 20 wt. % of the hydrophilic polymer chains do not comprise such a reactive group. More preferably at least 50 wt. %, still more preferably at least 80 wt. % of the hydrophilic polymer chains do not comprise such a reactive group. Most preferably the hydrophilic polymer chains do not comprise any of such reactive groups at all.

Reactive Diluents

The coating composition may comprise one or more reactive diluents, defined as a compound that has at least one group capable of reacting mutually and or capable of reacting with the reactive groups grafted to the particles.

In principle a wide variety of compounds are suitable to be used as the reactive diluent, for example monomers or oligomers having the same groups as the reactive groups as defined above. In a preferred embodiment, these reactive diluents are water soluble in the same temperature range as the grafted hydrophilic polymer.

Possible compounds that may be used as the reactive diluent are isocyanates, alkoxy titanates, alkoxy zirconates, or urea-, urea/melamine-, melamine-formaldehyde or phenol-formaldehyde (resol, novolac types), or radical curable (peroxide- or photo-initiated) unsaturated mono- and polyfunctional monomers and polymers, e.g. acrylates, methacrylates, maleate/vinyl ether), or radical curable (peroxide- or photo-initiated) unsaturated e.g. maleic or fumaric, polyesters in styrene and/or in methacrylates.

Method for Crosslinking.

Any cross-linking method that may cause the reactive groups to react and so to form the cross-linked phase so that a coating is formed is suitable to be used in the process according to the invention. Suitable ways to initiate crosslinking are for example electron beam radiation, electromagnetic radiation (UV, Visible and Near IR), thermally and by adding moisture, in case moisture curable compounds are used. In a preferred embodiment crosslinking is achieved by UV-radiation. The UV-crosslinking may take place through a free radical mechanism or by a cationic mechanism, or a combination thereof. In another preferred embodiment the crosslinking is achieved thermally. Also combinations of different cure methods are possible.

Initiator

An initiator may be present in the mixture to initiate the crosslinking reaction. The amount of initiator may vary between wide ranges. A suitable amount of initiator is for example between above 0 and 5 wt % with respect to total weight of the compounds that take part in the crosslinking reaction.

When UV-crosslinking is used, the mixture preferably comprises one or more UV-photo-initiators. Any known UV-photo-initiators may be used in the process according to the invention. For example; from Ciba, Darocur 1173 (2-Hydroxy-2-methyl-1-phenyl-1-propanone (CAS no. 7473-98-5)), 1- Irgacure 184 (Hydroxycyclohexyl phenyl ketone (CAS no. 947-19-3)), Irgacure 819 (Phenyl bis(2,4,6-trimethyl benzoyl) phosphine oxide (CAS no. 162881-26-7)), Irgacure 369 (2-Benzyl-2-(dimethylamino)-1-[4-(4-morpholinyl) phenyl}-1-butanone (CAS no. 119313-12-1)), Quanticure (EPD Ethyl 4-dimethylaminobenzoate (CAS no.10287-53-3)), Quanticure ITX (2-Isopropylthioxanthone (CAS no. 5495-84-1)), Benzophenone (CAS no. 119-61-9).

Coating Thickness

The coating according to the invention can be prepared in any suitable thickness, but it should be noted that thickness can also be a function of the amount of solids in the coating composition. The coatings according to the invention typically have a thickness ranging between 50 nm to tens of micrometers. Preferably the coating thickness is from 50 nm to 1000 nm, more preferably from 100 nm to 800 nm, even more preferably from 200 nm to 600 nm. The coating compositions preferably contain a weight percentage of solids from 2 wt % to 7 wt %.

Substrates

A wide variety of substrates may be used as a substrate in the process according to the invention. Suitable substrates are for example flat or curved, rigid or flexible substrates including films of for example polyolefins such as polyethylene (PE), low density polyethylene (LDPE), linear low density polyethylene (LLDPE), ultra high molecular weight polyethylene (UHMWPE), high density polyethylene (HDPE), crosslinked polyethylene (XLPE), polypropylene (PP), polymethylpentene (TPX), polybutylene (PB), polyisobutene (PIB), polystyrene (PS), polyvinyl chloride (PVC), polyvinylidene chloride (PVDC), polynorbornene. Polyarylates such as polymethyl methacrylate (PMMA), polymethyl acrylate (PMA), hydroxyethyl methacrylate (HEMA), polybutadiene acrylonitrile (PBAN), polyacrylamide (PAM), polyphenylene sulfide (PPS), polyphenylene ether (PPO). Polyesters such as polyethylene terephthalate (PET), polybutylene terephthalate (PBT), poly(cyclohexylene dimethylene terephthalate) (PCTA), polycyclohexylenedimethylene terephthalate glycol (PCTG), polyethylene terephtalate glycol (PETG), polytrimethylene terephthalate. Polysulphones such as polysulfone (PSU), polyarylsulfone (PAS), polyethersulfone (PES), polyphenylsulfone (PPS). Polyamides such as PA11, PA12, PA 66, PA6, PA46, PA6-co-PA66, PA610, PA69, polyphthalamide (PPA), bismaleimide (BMI), urea formaldehyde (UF). Cellulosics such as cellulose acetate, cellulose acetate butyrate, cellulose acetate propionate, ethyl cellulose, cellulose propionate. Polyurethanes such as polyurethane (PU), polyisocyanurate (PIR). Fluoropolymers such as fluoropolymer (FE), polytetrafluoroethylene (PTFE), ethylene chlorotrifluoroethlyene (ECTFE). Polycarbonate (PC), polylactic acid (PLA), polyimide, polyetherimide, polyetheretherketone (PEEK), polyetherketon (PEK), polyestercarbonate. Copolymers such as acrylonitrile butadiene styrene (ABS), ethylene vinyl acetate, ethylene vinyl alcohol, ethylene N-Butyl Acrylate, polyamide-imide or amorphous solids, for example glass or crystalline materials, such as for example silicon or gallium arsenide. Metallic substrates such as titanium and steel may also be used.

Preferred substrates include polypropylene, polyethylene, polystyrene, polyethylene terephthalate, polycarbonate, polyester, polyvinyl acetate, polyvinyl pyrollidone, polyvinyl chloride, polyimide, polyethylene naphthalate, polytetrafluoro ethylene, nylon, silicone rubber, polynorbornene, glass, titanium, steel, and combinations thereof.

The substrates are preferably able to be molded into, for example, biological sample (e.g. blood) collection tubes, microtitre plates, microfluid devices, biosensors, cell culture flasks and dishes, microtubes, PCR tubes, separation filters, and pipette tips.

A free-standing coating obtainable by a process according to the invention may be obtained by preparing a film or coating on a substrate and subsequently removing the film or coating from the substrate after crosslinking.

Application of the Mixture to a Substrate

The mixture may be applied onto the substrate by any process known in the art of wet coating deposition in one or multiple steps. Examples of suitable processes are spin coating, dip coating, spray coating, flow coating, meniscus coating, capillary coating and roll coating, aspiration coating, or suitable combinations thereof.

An object may be totally coated or partially coated with the coating composition. Also partial crosslinking of the coating and removal of the non-crosslinked part is possible, by for instance but not limited to photolithography.

In a first embodiment the mixture according to the invention is applied as the only coating on the substrate. In a second embodiment the coating in applied on top of one or more coatings. Those versed in the art will know which coatings to select to optimise properties such as adhesion, hardness, optical clarity etc.

After application and curing of the coating, further processing steps such as but not limited to a heat treatment or radiation treatment is possible.

Solvent

The composition according to the invention may comprise a solvent, for example to prepare a composition according to the invention that is suitable for application to the substrate using the chosen method of application.

In principle, a wide variety of solvents may be used. The solvent preferably has the ability to form stable suspensions of the particles grafted with the reactive groups and the hydrophilic polymer chains, in order to obtain good quality coatings i.e. after evaporation of the solvent. The particles typically are added to the mixture in the form of a suspension. The same solvent as used in the suspension may be used to adjust the mixture so that it has the desired properties. However, other solvents may also be used.

Examples of solvents that may be suitable are 1,4-dioxane, acetone, acetonitrile, chloroform, chlorophenol, cyclohexane, cyclohexanone, cyclopentanone, dichloromethane, diethyl acetate, diethyl ketone, dimethyl carbonate, dimethylformamide, dimethylsulphoxide, ethanol, ethyl acetate, m-cresol, mono- and di-alkyl substituted glycols, N,N-dimethylacetamide, p-chlorophenol, 1,2-propanediol, 1-pentanol, 1-propanol, 2-hexanone, 2-methoxyethanol, 1-methoxy-2-propanol, 2-octanone, 2-propanol, 3-pentanone, 4-methyl-2-pentanone, hexafluoroisopropanol, methanol, methyl acetate, methyl acetoacetate, methyl ethyl ketone, methyl propyl ketone, n-methylpyrrolidone-2, n-pentyl acetate, phenol, tetrafluoro-n-propanol, tetrafluoroisopropanol, tetrahydrofuran, toluene, xylene and water. Alcohols, ketones and esters based solvents may also be used, although the solubility of acrylates may become an issue with high molecular weight alcohols. Halogenated solvents (such as dichloromethane and chloroform) and hydrocarbons (such as hexanes and cyclohexanes), may also be suitable.

Preferably, the solvent used evaporates after applying the mixture onto the substrate. In the process according to the invention, optionally the mixture may after application to the substrate be heated or treated in vacuum to aid evaporation of the solvent.

Preferably the solvent has an evaporation rate (where butyl acetate equals 100) of from 100 to 200, more preferably from 110 to 190.

A list of evaporation rates for various solvents can be found in

“Selected evaporation rate and surface tension of solvents” P. C. Nicholas ed., Industrial Solvents Handbook, 2nd Ed., 2003, Marcel Dekker).

Preferably, solvents are used that give the coating composition a surface tension that is below 40 mN/m at 25° C., more preferable below 30 mN/m. The surface tension of the solvent(s) is preferably higher than 10 mN/m.

In a more preferred embodiment the solvent is selected from water, methanol, ethanol, isopropanol, n-propanol, butanol, isobutanol, acetone, methylether ketone, methylisobutyl ketone, isophorone, amyl acetate, butyl acetate, ethyl acetate, butylglycol acetate, butyl glycol, ethyl glycol, 2- nitropropane, and combinations thereof.

Most preferred solvents are water, methanol, ethanol, n-propanol, isopropanol and combinations thereof.

Adhesion Promoters

Preferably the composition according to the invention comprises a compound that increases the adhesion of the coating to the substrate. These may be for example silane acrylate compounds for usage of acrylate-containing coatings on glass. The skilled artisan will be able to select a suitable adhesion promoter for the desired substrate.

Additional Additives

In a further embodiment the composition according to the invention may contain one or more species that diffuse out of the coating during usage. Such species may be used for lubricity, adhesion purposes or comprise therapeutic species. Examples of such species are for instance but not limited to heparin, vitamins, anti-inflammatory agents, antimicrobial functionalities such as quaternary ammonium ions, peptide sequences, halogen labile species etc., biomolecule receptor sites.

Post-processing steps, after the composition has been applied to the substrate may include: addition of migratable species, for instance drugs, via reversible sorption, or chemical grafting of bioactive species to remnant reactive groups in the coating.

Applications

The invention also relates to a film or coating obtainable by the coating method according to the present invention. The invention also relates to substrates and articles partly or in whole coated with the coating composition obtainable according to the present invention.

Applications of the coating include coatings with anti-biofouling or anti-thrombogenic properties, coatings with anti-inflammatory properties, anti-microbial coatings, coatings to prevent biofilm formation, coatings for bioreceptors, coatings for biosensors, haemo-repellent coatings for blolod collection tubes and blood contact devices, coatings with anti-fogging properties. It is also possible that the coating is applied to an object to enhance wetting by aqueous solutions of the object.

The present coatings may be advantageously used for biological sample (e.g. blood) collection tubes, microtitre plates, microfluid devices, biosensors, cell culture flasks and dishes, microtubes, PCR tubes, separation filters, pipette tips, and the like. The present coatings may also be used for medical devices such as catheters, implants, stents, and the like. Preferred uses for the present coatings include blood collection tubes (e.g. Vacutainers®) and microtitre plates.

The invention also relates to a process for producing the coating composition according to the present invention comprising the step of chemically grafting a hydrophilic chain to a particle.

With this process various coating compositions may be obtained, suitable for all kind of applications.

The invention will be further explained by the examples, without being limited thereto.

### EXAMPLES

Synthesis of mPEG Trimethoxysilane (mPEG-Silane)

50 g (49.3 mmol) of polyethylene glycol mono methyl ether (mPEG) (Mw˜1100) was dissolved in 600 ml of toluene and the mixture was dried over night over 4 Å molecular sieves using soxhlet extraction (50° C./70 mbar). The concentration of mPEG in toluene was ˜8.0 wt./vol. %. After drying, 12.9 grams (52.2 mmol; 1.06 eq) triethoxy(3-isocyantopropyl)silane (Isocyante) was added drop wise to the reaction mixture. The amount of isocyanate was ˜6 mol.% excess with respect to the hydroxyl group of mPEG. The addition of isocyanate was done at room temperature. After addition, 8 drops (+/−65 mg) of dibutyltin dilaurate (DBTDL) catalyst was added to the stirring reaction. The reaction mixture was stirred continuously over night at room temperature, under nitrogen atmosphere. The reaction was monitored by FT-IR, following the disappearance of the NCO stretch frequency at 2270 cm⁻¹. After the reaction, approximately 80% of the toluene was removed by rotary evaporation and the mPEG-Silane was slowly precipitated into heptanes and washed several times with heptanes. The resulting white wax was dried in a vacuum oven at 50° C. over night. The product was checked by ¹H-NMR and GPC. Yield 90-95%; over 95% pure.

### Example 1

### Coating of a PET Blood Collection Tube Preparation of a Coating Composition

Silica dioxide particles suspended in methanol (MT-ST) were obtained from the Nissan Chemical America Corporation and surface modified by reaction with acryloxypropyltrimethoxysilane (APTMS, ABCR Chemicals) and mPEG-Silane (synthesised as described above) by the following method: A 1 liter 3-necked flask was charged with 30 wt. % MT-ST silica particles solution. The radical scavenger hydroquinone monomethyl ether and the initiator 1-hydroxycyclohexyl phenyl ketone were added. APTMS was added drop wise under stirring. Afterwards, a solvent was added. The mixture was heated to 70° C., and kept stirring for 2 hours. The mixture was allowed to cool down to room temperature.

Thereafter, mPEG-Silane was batch wise added to the mixture, and two molar equivalents of solvent to mPEG-Silane were added afterwards. The mixture was heated to 70° C., and kept stirring over night (for minimal 12 hours). After reaction, the mixture was allowed to cool down to room temperature and the functionalised particles solution was collected. This functionalised particles solution was used as a coating composition to coat a PET blood collection tube.

The coating compositions A-M according to tables 1-3 were prepared according to the above described method. The weight percentage (wt %) of solids in the coating compositions was 7 wt %. The surface tension of the coating compositions was determined according to J. Chem. Eng. Data, 1995, 40, 611-614.

Coating Procedure

Before coating, the polyethyleneterephthalate (PET) tubes used as the substrate were cut to a length of 45 mm, this to fit the glass measuring vials. After cutting all tubes were cleaned with methanol, rinsed with water and dried in a vacuum oven. A PET tube was fixed in place and filled with a coating formulation. After this the tube was aspirated using a thin metal tube connected to a vacuum desiccator. The dessicator was set to 600 mbar using a vacuum pump and provided a stable suction source to aspirate the tube. After aspiration of the coating formulation, the suction was maintained for another 10 seconds to allow complete aspiration of the tube. After coating the tube was kept under atmospheric conditions for 5-10 minutes and then flushed with nitrogen for 20-30 seconds before curing with UV light. The tube was cured with two times 5 seconds of UV exposure, which was similar to c.a. two times 1.0 J cm⁻².

TABLE 1
Compounds used for preparation of the coating compositions, in weight
percent, with various ratios of methanol/water as application solvent.

| Material | A | B | C | D | E |
|---|---|---|---|---|---|
| 30 wt. % MT-ST Silicon | 31.71% | 31.71% | 31.71% | 31.71% | 31.71% |
| oxide nano solution | | | | | |
| APTMS | 1.51% | 1.51% | 1.51% | 1.51% | 1.51% |
| mPEG-silane (Mw | 3.14% | 3.14% | 3.14% | 3.14% | 3.14% |
| 1100 g mol⁻¹) | | | | | |
| Hydroquinone | 0.01% | 0.01% | 0.01% | 0.01% | 0.01% |
| monoethyl ether | | | | | |
| Water | 0.18% | 0.18% | 0.18% | 0.18% | 0.18% |
| 1-hydroxycyclohexyl | 0.027% | 0.027% | 0.027% | 0.027% | 0.027% |
| phenyl ketone | | | | | |
| Methanol (solvent) | 0% | 15.85% | 31.71% | 47.57% | 63.42% |
| Water (solvent) | 63.42% | 47.57% | 31.71% | 15.85% | 0% |
| Ratio of water/methanol | 100% | 75% | 50% | 25% | 0% |

TABLE 2
Compounds used for preparation of the coating compositions, in weight
percent, with various ratios of ethanol/water as application solvent.

| Material | F | G | H | I |
|---|---|---|---|---|
| 30 wt. % MT-ST Silicon | 31.71% | 31.71% | 31.71% | 31.71% |
| oxide nano solution | | | | |
| APTMS | 1.51% | 1.51% | 1.51% | 1.51% |
| mPEG trimethoxysilane | 3.14% | 3.14% | 3.14% | 3.14% |
| (Mw 1100 g mol⁻¹) | | | | |
| Hydroquinone | 0.01% | 0.01% | 0.01% | 0.01% |
| monoethyl ether | | | | |
| Water | 0.18% | 0.18% | 0.18% | 0.18% |
| 1-hydroxycyclohexyl | 0.027% | 0.027% | 0.027% | 0.027% |
| phenyl ketone | | | | |
| Ethanol (solvent) | 15.85% | 31.71% | 47.57% | 63.42% |
| Water (solvent) | 47.57% | 31.71% | 15.85% | 0% |
| Ratio of water/ethanol | 75% | 50% | 25% | 0% |

TABLE 3
Compounds used for preparation of the coating compositions, in weight
percent, with various ratios of isopropanol/water as application solvent.

| Material | J | K | L | M |
|---|---|---|---|---|
| 30 wt. % MT-ST Silicon | 31.71% | 31.71% | 31.71% | 31.71% |
| oxide nano solution | | | | |
| APTMS | 1.51% | 1.51% | 1.51% | 1.51% |
| mPEG trimethoxysilane | 3.14% | 3.14% | 3.14% | 3.14% |
| (Mw 1100 g mol⁻¹) | | | | |
| Hydroquinone monoethyl | 0.01% | 0.01% | 0.01% | 0.01% |
| ether | | | | |
| Water | 0.18% | 0.18% | 0.18% | 0.18% |
| 1-hydroxycyclohexyl | 0.027% | 0.027% | 0.027% | 0.027% |
| phenyl ketone | | | | |
| Isopropanol (solvent) | 15.85% | 31.71% | 47.57% | 63.42% |
| Water (solvent) | 47.57% | 31.71% | 15.85% | 0% |
| Ratio of water/Isopropanol | 75% | 50% | 25% | 0% |

¹²⁵I-BSA Adsorption Testing in PET Tubes

Radioactively labeled BSA was used to evaluate the coating performance when applied to the substrate. Before starting the performance evaluation of the tubes a buffer solution was prepared to dilute the ¹²⁵I-BSA and obtain a radioactively labeled protein solution with a desired activity of about 74 kBq/ml. After this the solution was ready to be used for testing.

Preparation of Buffer Solution

To a 1l volumetric flask was added:

−900 ml of demi water

−6.96 g K₂HPO₄

−1.56 g NaH₂PO₄*2H₂O

−8.76 g NaCl

−10.0 mg BSA (not labeled)

After this the pH was adjusted to 7.4±0.1 with 1 M NaOH and the volume was adjusted to 1l.

¹²⁵I-BSA Solution:

The buffer solution was used to dilute the radioactive labeled ¹²⁵I-BSA (purchased from Perkin Elmer) to ±74 kBq/ml. The amount of dilution depended on the activity of the original material.

1 ml of ¹²⁵I-BSA solution was added to the tube. The tube was allowed to stand overnight (˜20 hours) at room temperature. After this the tube was emptied using a pipette, followed by 3 washing steps with demi water. The tube was put into a LSC vial. The tube was then filled with 2 ml Pico-Fluor 15 (Scintillation cocktail) and the space around the tube was filled with ˜18 ml Pico-Fluor 15. The vial was closed and the activity was measured using a scintillation counter. The percentage of protein absorption was calculated by the number of residual counts compared to a blanc measurement. ¹²⁵I-BSA absorption gives a value for the amount of protein (albumin) absorption on the coated surface and is thus a measure for the occurence of biofouling on a surface.

A ¹²⁵I-BSA absorption below 20% is considered a good performance of the coating composition.

A ¹²⁵I-BSA absorption below 10% is considered an excellent performance of the coating composition.

TABLE 4
Reduction in ¹²⁵⁻I BSA absorption of the various alcohol/water
mixtures and the corresponding surface tensions of coating solvents.

| | Tube coated | | Surface | |
|---|---|---|---|---|
| | with various | | tension | ¹²⁵⁻I BSA |
| Formulation | water/alcohol | | mN/m | absorption |
| code | mixture | | @ 25° C. | % |
| | Uncoated tube | | 47 | 100 |
| A | 100% | water | 72.01 | 28.6 |
| B | 25% | Methanol | 43.78 | 33.3 |
| C | 50% | Methanol | 32.86 | 7.6 |
| D | 75% | Methanol | 26.51 | 2.5 |
| E | 100% | Methanol | 22.51 | 2.5 |
| F | 25% | Ethanol | 35.51 | 15.6 |
| G | 50% | Ethanol | 27.96 | 10.1 |
| H | 75% | Ethanol | 24.42 | 10 |
| I | 100% | Ethanol | 21.82 | 5.5 |
| J | 25% | Isopropanol | 28.28 | 12.4 |
| K | 50% | Isopropanol | 24.26 | 7.7 |
| L | 75% | Isopropanol | 22.41 | 10.1 |
| M | 100% | Isopropanol | 21.22 | 3 |

### Example 2

### Coating Performance in Microtiter Plates

¹²⁵I BSA absorption in coated polystyrene microtiter plates was determined in relation to the concentration of solids in the coating solution.

The relation between the concentration of solids in the coating solution and reduction in ¹²⁵I BSA absorption was examined in coated polystyrene microtiter plates. This was done by applying a coating solution with 2 wt %, 3 wt % and 7 wt % of solids inside the wells of a microtiter plate using a 96 well plate washer (BioTek Elx-405) to control the coating application and aspiration process. The coated plates were allowed to dry for 5 minutes at 23° C. and 55% RH before curing with UV light. The coating was cured under nitrogen inertion with 2 times 1.0 J cm⁻² of UV. The coating performance was evaluated by using a radioactively labelled protein solution to determine the reduction in protein adsorption.

The buffer solution and the¹²⁵I-BSA solution were prepared as described above. 100 μl of ¹²⁵I-BSA solution was added to the wells of a microtiter plate. The plate was allowed to stand overnight (˜20 hours) at room temperature. After this the plate was emptied using a pipette, followed by 3 washing steps with demi water. The plate was allowed to dry for 30 minutes before measuring the residual activity with a contamination counter (Geiger counter). The reduction in protein adsorption was calculated by the number of residual counts compared to an uncoated microtiter plate.

The results can be found in table 6. This shows that the coating applied from the 7 wt. % solution shows a lower protein adsorption (<5%) and greater mechanical stability compared to coating applied from the 2 wt. % solution (˜50%). However, the residual structure is worse for the 7 wt. % solution and this coating cracks after prolonged exposure to water.

TABLE 5
Compounds used for preparation of the coating
formulation with various solid concentrations

| | Concentration of solids (wt. %) | | |
|---|---|---|---|
| Material | 2% | 3% | 7% |
| 30 wt. % MT-ST Silicon | 10.74% | 15.56% | 31.71% |
| oxide nano solution | | | |
| Acr-Pr-TMS | 0.51% | 0.74% | 1.51% |
| mPEG trimethoxysilane | 1.06% | 1.54% | 3.14% |
| (Mw 1100 g mol⁻¹) | | | |
| Hydroquinone monoethyl ether | 0.003% | 0.005% | 0.01% |
| (inhibit polymerisation) | | | |
| 1-hydroxycyclohexyl | 0.009% | 0.013% | 0.027% |
| phenyl ketone (initiator) | | | |
| Water | 0.06% | 0.09% | 0.18% |
| n-propanol (solvent) | 87.61% | 82.06% | 63.42% |

TABLE 6
Coating composition related ¹²⁵⁻1 BSA absorption,
coating structure, and water cracking resistance.

| | Concentration of solids wt. % | | |
|---|---|---|---|
| Evaluation | 2% | 3% | 7% |
| ¹²⁵⁻I BSA absorption % | 45~50% | 7% | <5% |
| Residual Structure | Good | Good | Bad |
| Water cracking | No | No | Yes |
| Mechanical properties | Poor | Medium | Good |
| (scratching resistance) | | | |

1.-13. (canceled)

14. Method for providing a substrate with an anti-biofouling coating the method comprising:
a. obtaining a coating composition comprising nanoparticles being grafted with reactive groups and hydrophilic polymer chains and a solvent;
b. applying the coating composition to the substrate; and
c. optionally curing the coating composition.
wherein the surface tension of the coating composition at 25° C. is below 40 mN/m.

15. Method according to claim 14 wherein the surface tension of the coating composition is below 30 mN/m.

16. Method according to claim 14 wherein the surface tension of the coating composition is higher than 10 mN/m.

17. Method according to claim 14 wherein the weight percentage of solids in the coating composition is from 2 wt % to 7wt %.

18. Method according to claim 14 wherein the reactive group is selected from acrylates, methacrylates, epoxy, vinyl ethers, allyl ethers, styrenics, or combinations thereof.

19. Method according to claim 14 wherein the hydrophilic polymer chain comprises monomer units of ethylenoxide, (meth)acrylic acid, (meth)acrylamide, vinylpyrrolidone, 2-hydroxyethyl(meth)acrylate, phosphorylcholine, glycidyl(meth)acrylate or saccharides.

20. Method according to claim 14 wherein the nanoparticles comprise SiO₂.

21. Method according to claim 14 wherein the coating composition also comprises a reactive diluent.

22. Method according to claim 14 wherein the coating composition comprises a reactive diluent that is water soluble in the same temperature range as the grafted hydrophilic polymer.

23. Method according to claim 14 wherein the coating composition comprises a UV-photoinitiator.

24. Method according to claim 14 wherein the coating composition comprises a solvent selected from water, methanol, ethanol, isopropanol, n-propanol, butanol, isobutanol, acetone, methylether ketone, methylisobutyl ketone, isophorone, amyl acetate, butyl acetate, ethyl acetate, butylglycol acetate, butyl glycol, ethyl glycol, 2- nitropropane, and combinations thereof.

25. Method according to claim 14 wherein the coating composition is applied to the substrate by spin coating, dip coating, spray coating, flow coating, meniscus coating, capillary coating and roll coating, aspiration coating, or suitable combinations thereof.

26. A substrate coated according to claim 14.

27. A substrate according to claim 26 wherein the substrate is selected from polypropylene, polyethylene, polystyrene, polyethylene terephthalate, polycarbonate, polyester, polyvinyl acetate, polyvinyl pyrollidone, polyvinyl chloride, polyimide, polyethylene naphthalate, polytetrafluoro ethylene, nylon, silicone rubber, polynorbornene, glass, titanium, steel, and combinations thereof.

28. A molded article coated according to claim 14 wherein the article is selected from biological sample (e.g. blood) collection tubes, microtitre plates, microfluid devices, biosensors, cell culture flasks and dishes, microtubes, PCR tubes, separation filters, and pipette tips.
